(19) United States
(12) Patent Application Publication
(10) Pub. No.: US 2008/0260873 A1
(43) Pub. Date: Oct. 23, 2008

(54) Agent for prevention or improvement of neuropathy comprising pre-germinated brown rice lipid fraction as an effective ingredient

(75) Inventors: Seigo Usuki, Augusta, GA (US); Robert K. Yu, Augusta, GA (US); Yukihiko Ito, Yokohama-shi (JP); Keiko Morikawa, Yokohama-shi (JP); Mitsuo Kise, Yokohama-shi (JP)

Correspondence Address: YOUNG & THOMPSON, 209 Madison Street, Suite 500, ALEXANDRIA, VA, 22314, US

(73) Assignee: FANCL CORPORATION, KANAGAWA (JP)

(21) Appl. No.: 11/785,978

(22) Filed: Apr. 23, 2007

(51) Int. Cl. A61K 36/899; A61P 25/00; A61P 25/02

(52) U.S. Cl. 424/750

(57) ABSTRACT

The present invention is intended to find a novel function of a component in pre-germinated brown rice and to provide a safe and effective agent or functional food for prevention or improvement of a neuropathy or diabetic neuropathy

The present invention provides an agent or functional food for prevention or improvement of a neuropathy or diabetic neuropathy including a total lipid fraction of pre-germinated brown rice as an effective ingredient.

### BACKGROUND OF THE INVENTION

1. Field of the Invention

The present invention relates to a use of a lipid fraction in pre-germinated brown rice, and more specifically to a use of a lipid fraction in pre-germinated brown rice for prevention or improvement of diabetic neuropathy.

2. Description of the Related Art

According to statistics in 2000, the number of diabetic patients is 84.5 million in Asia and 151 million in the world. In Japan, according to diabetes survey by the Health, Labour and Welfare Ministry in 2002, the number of diabetic patients and potential diabetic patients is 16.2 million, that is, one out of every 6.3 adults suffers from diabetes. Meanwhile, the number of patients is likely to increase to 132.3 million in Asia and 221 million in the world in 2015 (see Non-Patent Document 1).

Diabetes mellitus (DM) develops due to abnormality of sugar metabolism and causes or may cause various typical complications by a morbid increase in a blood glucose level. The term “complications” as used herein refers to diseases or symptoms caused by a certain disease. Diabetes itself does not cause severe subjective symptoms, and in many cases, patients with diabetes do not receive treatment before complications appear, resulting in worsening clinical conditions.

Examples of the complications of diabetes include cerebral infarction, stroke, myocardial infarction, diabetic nephropathy, lower limb arteriosclerosis obliterans, diabetic retinopathy, dermatosis, infectious diseases, diabetic neuropathy, hyperlipidemia, and vascular dementia. Among them, diabetic nephropathy, diabetic retinopathy, and diabetic neuropathy are referred to as three major complications.

Diabetes is caused by genetic factors in some cases but mainly by lifestyle such as eating habit, and therefore expectations for health foods and functional foods are increasingly raised. In recent years, usefulness of pre-germinated brown rice for the complications of diabetes have attracted attention, and various effects have been reported, such as the effect of improving hyperlipidemia, the effect of preventing cardiovascular diseases (suppressing thrombus formation), and the effect of preventing diabetic nephropathy.

The term “pre-germinated brown rice” as used herein refers to germinating brown rice, where the size of a germ is less than 1 mm. It can produce γ-aminobutyric acid (GABA), which is known to have antihypertensive action and antistress action, in germination process. Moreover, pre-germinated brown rice contains rich dietary fibers, vitamins, minerals, and unknown lipids in the bran layer or germ and is popular in Japan as a new whole-grain cereal and as a subject of study for use as a principal food. Pre-germinated brown rice has been proven to be useful for health purposes. It has been reported in an animal study that pre-germinated brown rice has an effect of lowering the blood glucose level of streptozotocin (STZ)-induced diabetic rats (see Non-patent Document 2). Meanwhile, compared to white rice, a diet of pre-germinated brown rice is known to lower the blood glucose level and the insulin level after eating among healthy subjects (see Non-Patent Document 3) and patients with hyperglycemia (see Non-Patent Document 4) and is appreciated to be useful as a principal food for preventing diabetes.

As described above, the improving effect of pre-germinated brown rice on diabetes has been examined, but an effect on diabetic neuropathy, which is one of three major complications of diabetes, has not been studied yet. Therefore, the inventors of the present invention examined al efficacy of the intake of pre-germinated brown rice on diabetic neuropathy using STZ-induced diabetic rats, compared with brown rice and white rice.
[Non-Patent Document 1] Zimmet P, Alberti K G, Shaw J. Global and societal implications of the diabetes epidemic. Nature. Dec. 13, 2001;414(6865):782-7. Review
[Non-Patent Document 2] Hagiwara H, Seki T, Ariga T. The effect of pre-germinated brown rice intake on blood glucose and PAI-1 levels in streptozotocin-induced diabetic rats. Biosci Biotechnol Biochem. February 2004;68(2):444-7.
[Non-Patent Document 3] Ito Y, Mizukuchi A, Kise M, Aoto H, Yamamoto S, Yoshihara R. Yokoyama J. Postprandial blood glucose and insulin responses to pre-germinated brown rice in healthy subjects. J Med Invest. August 2005;52(3-4):159-64.
[Non-Patent Document 4] Ito Y, Shen M, Kise M, Hayamizu K, Yoshino G, Yoshihara R, Yokoyama J: Effect of pre-germinated brown rice on postprandial blood glucose and insulin level in subjects with hyperglycemia. Jpn J Food Chem 2005;12(2):80-4.

### SUMMARY OF THE INVENTION

Pre-germinated brown rice has been conventionally used as a health food, and therefore it may be provided as a pharmaceutical or food that is quite harmless and can be adopted for a long period of time. Moreover, in recent years, attention has been focused on the efficacy of pre-germinated brown rice, such as the effect of improving hyperlipidemia, the effect of preventing cardiovascular diseases (suppressing thrombus formation), and the effect of preventing diabetic nephropathy.

The inventors of the present invention discovered in the preceding experiments that pre-germinated brown rice has an effect of lessening the severity of diabetic neuropathy that is one of three major complications of diabetes. It is beneficial to specify and use an ingredient that is contained in pre-germinated brown rice and has an effect of improving the neuropathy for health maintenance or promotion of human or animals, prevention or treatment of diseases, and the like, and the object of the present invention is to discover a novel function of the ingredient contained in pre-germinated brown rice.

The inventors of the present invention have made extensive studies to solve the above-mentioned problems, and as a result, they have discovered that a lipid fraction contained in pre-germinated brown rice or a bran layer of pre-germinated brown rice has an effect of improving diabetic neuropathy, thereby achieving the present invention.

That is, the present invention includes the followings.
(1) An agent for prevention or improvement of a neuropathy including a pre-germinated browm rice lipid fraction as an effective ingredient.
(2) Am agent according to Item (1), in which the pre-germinated brown rice lipid fraction is a chloroform-methanol soluble component of pre-germinated brown rice.
(3) An agent according to Item (1), in which the neuropathy is diabetic neuropathy.
(4) An agent according to Item (1), in which the neuropathy is damage of myelinated nerves.
(5) An agent according to Item (1), in which the neuropathy is a decrease in activity of Na,K-ATPase derived from the nerve membrane.
(6) An agent according to Item (1), in which the neuropathy is accompanied by a decrease in activity of HTase in HDL derived from serum.
(7) A functional food for prevention or improvement of a neuropathy including a pre-germinated brown rice lipid fraction as an effective ingredient.
(8) A functional food according to Item (7), in which the pre-germinated brown rice lipid fraction is a chloroform-methanol soluble component of pre-germinated brown rice.
(9) A functional food according to Item (7), in which the neuropathy is diabetic neuropathy.
(10) A functional food according to Item (7), in which the neuropathy is damage of myelinated nerves.
(11) A functional food according to Item (7), in which the neuropathy is a decrease in activity of Na,K-ATPase derived from the nerve membrane.
(12) A functional food according to Item (7), in which the neuropathy is accompanied by a decrease in activity of HTase in HDL derived from serum.
(13) A method of preventing or treating a neuropathy including administering a pre-germinated brown rice lipid fraction.
(14) A method according to Item (13), in which the pre-germinated brown rice lipid fraction is a chloroform-methanol soluble component of pie-germinated brown rice.
(15) A method according to Item (13), in which the neuropathy is diabetic neuropathy.
(16) A method according to Item (13), in which the neuropathy is damage of myelinated nerves.
(17) A method according to Item (13), in which the neuropathy is a decrease in activity of Na,K-ATPase derived from the nerve membrane.
(18) A method according to Item (13), in which the neuropathy is accompanied by a decrease in activity of HTase in HDL derived from serum.

A lipid extract or a lipid fraction of the present invention, which is obtained from pre-germinated brown rice or a bran layer of pre-germinated brown rice, has an effect of improving diabetic neuropathy.

Therefore, according to the present invention, the extract or fraction can be used as an agent for prevention or improvement of diabetic neuropathy.

In addition, it is very safe, can be ingested continuously, produced in a large quantity, and easily added to food or the like. Accordingly, pre-germinated brown rice is likely to contribute to health promotion and disease prevention for human or animals.

### BRIEF DESCRIPTION OF THE DRAWINGS

In the accompanying drawings:

FIGS. 1A and 1B are graphs illustrating body weights (A) and blood glucose levels (B) of diabetic rats fed with a pre-germinated brown rice feed (DPR, n=9), a brown rice feed (DBR, n=9), and a white rice feed (DWR, n=9), and normal rats fed with a pre-germinated brown rice feed (PR, n=6), a brown rice feed (BR, n=6), and a white rice feed (WR, n≃6), where the respective values represent means±standard errors of the means;

FIGS. 2A1 to 2A3, 2B1 to 2B3, and 2C1 to 2C3 are histograms illustrating myelinated fibers (A1, B1, C1), myelinated axons (A2, B2, C2), and G ratios (A3, B3, C3), where (A): normal group fed with a control feed (AIN93G) (C, n=4), (B): diabetic group fed with a control feed (AIN93G) (DC, n=4), and (C): diabetic group fed with a pre-germinated brown rice feed (DPR, n=4);

FIGS. 3A and 3B are graphs illustrating correlations between HTase activity and Na,K-ATPase activity in (A): diabetic group fed with a brown rice feed (DBR) and in (B): diabetic group fed with pre-germinated brown rice feed (DPR), where regression lines determined by analyses of all the data points (n=9) are shown (a Pearson correlation coefficient was used to evaluate a simple linear relationship among variables);

FIG. 4A is a graph illustrating an effect of Hcy-thiolactone-modified LDL on Na,K-ATPase activity, where bar 1: no additive; bar 2: LDL; bar 3: LDL+Hcy-thiolactone; bar 4: LDL+Hcy-thiolactone+TLb (0.1 μg); bar 5: LDL+Hcy-thiolactone+TLb (1.0 μg); bar 6: LDL+Hcy-thiolactone+TLb (10 μg); bar 7: LDL+Hcy-thiolactone+TLp (0.1 μg); bar 8: LDL+Hcy-thiolactone+TLp (1.0 μg); bar 9: LDL+Hcy-thiolactone+TLp (10 μg), and the respective values represent means±standard errors of the means (n=6) (means of the values of the bars indicated by different characters (a, b, c, and d) were significantly different from each other (P<0.05)); and

FIG. 4B is a graph illustrating an effect of a lipid fraction (TLb or TLp) on HTase activity, where bar 1: no additive; bar 2: TLb (0.1 μg); bar 3: TLb (0.5 μg); bar 4: TLb (1.0 μg); bar 5: TLb (5.0 μg); bar 6: TLp (0.1 μg); bar 7: TLp (0.5 μg); bar 8: TLp (1.0 μg); bar 9: TLp (5.0 μg), and the respective values represent means±standard errors of the means (n=6) (means of the values of bars indicated by different characters (a, b, c, d, e, and f) were significantly different from each other (P<0.05)).

### DETAILED DESCRIPTION OF THE PREFERRED EMBODIMENTS

An effect of preventing or improving neuropathy of the present invention is provided by a lipid fraction extract of pre-germinated brown rice obtained from a raw material including at least pre-germinated brown rice. Pre-germinated brown rice can be prepared by known methods (see JP 2001-352916 A, JP 2002-136263 and JP 2002-360192 A).

The term “lipid fraction of pre-germinated brown rice” as used herein primarily refers to a total lipid fraction used in Referential Examples and Examples, but is not limited thereto. In addition, in any application for patent based on the present application for the priority, the definition of the lipid fraction of pre-germinated brown rice is not limited to that of the present application.

The total lipid fraction of pre-germinated brown rice is known to contain glycolipids, phospholipids, sterols, sphingolipids, γ-oryzanol, ferulic acid, hydrophobic proteins, etc. The total lipid fraction cm be used as a lipid fraction, but may be a lipid fraction including a single substance or a mixture of a plurality of substances, which can be obtained by further fractionation.

In order to extract the lipid fraction, volatile organic solvents or alcohols, or a mixture thereof may be used. It is effective to use chloroform as an organic solvent and methanol as an alcohol, but the substances are not limited thereto. For example, the mix ratio of the both substances is preferably 1:1 to 2:1 (volume ratio) (chloroform content: 50% or more to 67% or less), but is not limited thereto. Pre-genninated brown rice (5 g) is subjected to extraction once with 30 ml of a chloroform/methanol mixture (volume ratio 1:1), and then to further extraction with 20 ml of a chloroform/methanol mixture (volume ratio 2:1), and the both extracts are mixed. Then, the solvent is distilled away, and the residue is dried, to thereby yield a lipid fraction.

The resultant lipid fraction may be used without further modification, but may be purified to a single-ingredient lipid molecule or lipid molecular species by a separation method such as silica gel column chromatography, ion exchange column chromatography, or high performance liquid chromatography.

An extract obtained by the above-mentioned method may be used without further modification as a lipid fraction of the present invention. However, in general, the extract is dissolved or dispersed in an appropriate liquid, or mixed with or adsorbed to an appropriate powder carrier, and in some cases, an emulsifier, dispersant, suspending agent, spreader, penetrant humectant stabilizer, or the like is added thereto to use it as a pharmaceutical in the form of an emulsion, oil solution, hydration agent, powder, tablet, capsule, liquid, or the like.

In the case of use of the extract as a pharmaceutical, the amount thereof is different depending on the form of the pharmaceutical, but should be an effective dose, and the upper limit is not particularly specified because the amount has no influence on the safety.

Meanwhile, the term “functional food” as used herein refers to a food which includes a lipid fraction of pre-germinated brown rice as an effective ingredient and has the function to prevent or improve a neuropathy, or a food which is expected to exhibit such a function when the food is ingested, and includes a health food, a Food for Specified Health Use, and a Food with Nutrient Function Claims.

Examples of the food include: a sweet stuff such as a chewing gum, a candy, a sweet tablet, a gumi-jelly, a chocolate, a biscuit, or a snack; a frozen dessert such as an ice cream, a sherbet, or an ice candy; a soft drink; a pudding; a jam; a dairy product; and a flavoring; which can be ingested on a daily basis. The amount of a lipid fraction of the present invention to be added to these foods varies depending on the forms of the foods, but it is not necessary that the upper limit be specified because the amount has no influence on the safety.

The term “diabetic neuropathy” as used herein refers to a peripheral neuropathy or an autonomic neuropathy, which is one of the complications caused by diabetes. It causes clinical symptoms of numbness or pain in a limb, for example, (in an early stage) and sensory paralysis and ataxia (in a chronic stage). Pathological symptoms include degeneration and damage of nerve tissues such as myelin sheath and axon. Such neuropathy can be observed and quantified as a decrease in peripheral nerve conduction velocity or a decrease in activity of Na,K-ATPase (sodium-potassium ATPase) derived from nerve axon membrane. Ingestion of the lipid fraction of pre-germinated brown rice of the present invention provides an effect of improving diabetic neuropathy. The term “effect of improving diabetic neuropathy” as used herein refers to an effect provided by a lipid fraction of pre-germinated brown rice, that is, an effect of increasing Na,K-ATPase activity or HTase activity decreased by diabetic neuropathy to a near normal level or an effect of preventing a decrease in motor nerve conduction velocity, within a whole-body, a tissue, a cell or body fluid of human or animals. The term “HTase” as used herein refers to a hydrolase of homocysteine thiolactone, which is a risk factor for arteriosclerosis and is classified as the paraoxonase family. It has been reported that a patient suffering from a neuropathy has a decreased paraoxonase activity (see Abbott C A, Mackness M I, Kumar S, Boulton A J, Durrington P N. Serum paraoxonase activity, concentration, and phenotype distribution in diabetes mellitus and its relationship to serum lipids and lipoproteins. Aterioscler Thromb Vasc Biol. November 1995; 15(11) 1812-8), and therefore a decrease in HTase activity is considered not only to provide a risk factor for arteriosclerosis but also to be associated with the degree of progression of a neuropathy.

The term “neuropathy” as used herein refers to a clinical condition that exhibits physiological, cytohistological, or biochemical findings which are the same as or similar to that of diabetic neuropathy, but is not limited to a condition caused by diabetes. That is, it includes all neuropathies that can be observed and quantified as a damage of myelinated nerves, in particular, myelinated axons, a decrease in motor nerve conduction velocity, a decrease in activity of Na,K-ATPase derived from the nerve membrane, or a decrease in HTase activity in HDL derived from serum.

Specifically, the effect of improving a neuropathy of a lipid fraction of the present invention can be observed by, as shown in Referential Examples 1 to 4 below, electrophysiological measurement (in vivo) of motor nerve conduction velocity in tail nerve of a rat individual and pathological evaluation of nerve tissues, or, as shown in Examples 1 and 2, by an experiment (in vitro), for example, to examine Na,K-ATPase activity which is an index of diabetic neuropathy using sciatic nerves of diabetic rats.
[Patent Document 1] JP 2001-352916 A
[Patent Document 2] JP 2002-136263 A
[Patent Document 3] JP 2002-360192 A
[Non-Patent Document 5] Abbott C A, Mackness M I, Kumar S, Boulton A J, Durrington P N. Serum paraoxonase activity, concentration, and phenotype distribution in diabetes mellitus and its relationship to serum lipids and lipoproteins. Arterioscler Thromb Vasc Biol. November 1995;15(11):1812-8.

### REFERENTIAL EXAMPLE 1

Experiment for Comparison of Body Weights and Blood Glucose Levels of Rats Fed with Pre-Germinated Brown Rice, Brown Rice, or White Rice

(Experimental Materials and Methods)

Design of Animal Experiments

Rats were injected intraperitoneally with STZ and fed with a control feed (AIN93G) for two weeks. The diabetic rats were divided into three groups depending on feeds (white rice, brown rice, and pre-germinated brown rice). Normal rats were also divided into three groups. Thereafter, all the rats were allowed to freely ingest the experimental feeds and water for three weeks. At the end of the experiment, nerve conduction velocities were measured in the tails of the rats to evaluate neuropathy, and then the sciatic nerves were removed, followed by a pathological morphometric test and a Na,K-ATPase activity test. At the same time, total serum was collected and analyzed biochemically. Hereinafter, a group of normal rats fed with only the control feed and a group of diabetic rats fed with only the control feed were abbreviated as group C and group DC, respectively. In addition, groups of normal rats fed with white rice, brown rice, and pre-germinated brown rice were abbreviated as groups WR, BR, and PR, respectively, while groups of diabetic rats fed with white rice, brown rice, and pre-germinated brown rice were abbreviated as groups DWR, DBR, and DPR, respectively.

STZ-Diabetic Rats and Experimental Feeds

Male Wistar rats (body weight 120 to 140 g) were injected intraperitoneally with STZ (65 mg/kg, dissolved in sodium citrate (100 mM, pH 4.5)). One week after injection, blood samples were taken by tail puncture. Blood glucose levels were measured using a blood glucose meter (Accu-Check Advantage Blood Glucose Meter, Roche Diagnostics, Indianapolis, Ind.). Measurement for each experiment group was carried out regularly once a week after 22-hour fasting.

The rats were fed in a controlled environment, and the experiments were carried out in accordance with the guideline on use of experimental animals published by Medical College of Georgia.

All the rice feeds and control feed were manufactured by Harlan Teklad (Madison, Wis.) as feed powders. The control feed (AIN93G) was made from 39.7% corn starch, 13.2% α-corn starch, 20.0% casein, 0.3% L-cysteine, 10% sucrose, 7.0% soybean oil, 5.0% cellulose powder, 3.5% mineral mix, 1.0% vitamin mix, 0.25% choline bicitrate, and 0.0014% butylhydroquinone. The feed of pre-germinated brown rice, brown rice, or white rice was prepared by replacing corn starch and α-corn starch with pre-germinated brown rice, brown rice, or white rice.

Statistical Analysis

Multiple comparisons among the feed groups of the diabetic rats and the feed groups of the normal rats were carried out by one-way analysis of variance (ANOVA), and statistical differences were evaluated by the Tukey test for the parametric case or by the Kruskal-Wallis test for the nonparametric case. A p-value equal to or less than 0.05 was considered to be statistically significant. Moreover, comparative analyses of two groups were carried out (between group DC and the other feed groups in the diabetic rat group or between group C and the other feed groups in the normal rat group (by the Dunnett's test for the parametric case or by the Dunnett's test fox the nonparametric case)). Data are expressed as means±standard errors of the means (SEM).

(Results)

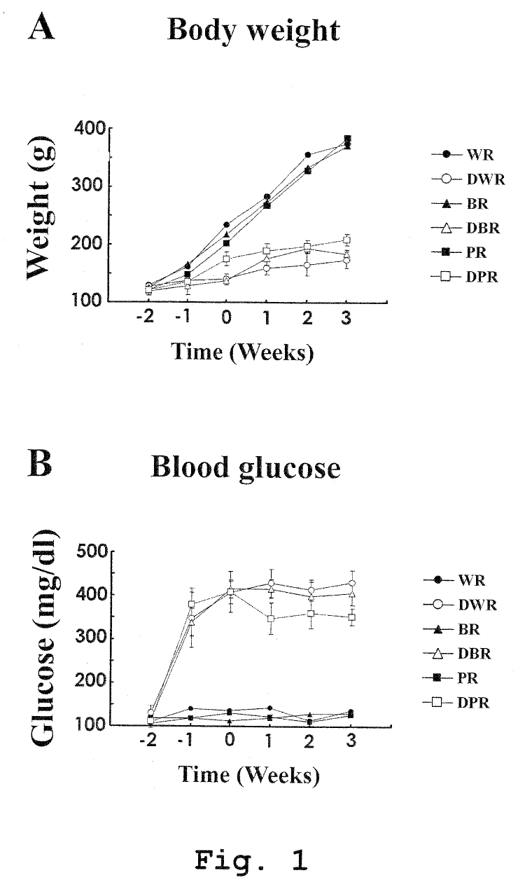

The results revealed that the body weights of the rats of groups WR, BR, and PR increased smoothly, and there were no significant differences among the ingested feeds (FIG. 1B). For DWR, DBR, and DPR groups, moderate body weight increases caused by diabetes were observed. In the case of the normal rats, there were no significant differences in increases in body weights between the rice feed ingestion groups (groups WR, BR, and PR) and the control ingestion feed group (group C). However, in the case of the diabetic rats, body weights of the rats of group DPR statistically significantly increased compared to group DC (P<0.01, Dunnett's test). For group C and all the rice feed groups (WR, BR, and PR), blood glucose levels were within a normal range over the experimental period (FIG. 1A, not shown for the group of normal rats fed with a control feed (group C)). On the other hand, in the cases of group DC and the rice feed groups of the diabetic rats (DWR, DBR, and DPR), blood glucose levels were found to increase (not shown for group DC). The blood glucose levels in the rats of group DPR were high for the first three weeks, but they were significantly lowered compared to groups DWR and WBR (p<0.05, Tukey test).

(Discussion)

As previously reported, the inventors of the present invention have discovered that the feeds of pre-germinated brown rice and brown rice can decrease blood glucose levels and that the feed of pre-germinated brown rice has a significantly high effect on diabetes compared to the feed of brown rice.

### REFERENTIAL EXAMPLE 2

Experiment for Comparison of Improvement in a Peripheral Neuropathy of Rats Fed with Pre-Germinated Brown Rice, Brown Rice, or White Rice

(Experimental Materials and Methods)

Tail Nerve Conduction Velocity

A method reported by Anderson et al. was modified and used for measurement of motor nerve conduction velocities (NCVs) in tail nerves of rats. The modification is in the way of stimulation where digital ring electrodes with twist wire were used instead of needle electrodes (Medtronic Functional Diagnostics, Skovlunde, Denmark). Briefly, an electrode for measurement was wound at a position 6 cm away from the base of the tail, and electrodes for stimulation were wound at positions 2 cm and 5 cm away from the electrode for measurement toward the base. The tail was electrically stimulated at the two positions separately to measure arrival time of electrical current. The NCVs were measured while the surface temperature of the tail was maintained at 34 to 35° C.

Preparation of Crude Sciatic Nerve Membrane for Measurement of Na,K-ATPase Activity

The crude membrane was prepared by a previously reported procedure (see Non-Patent Document 6). Briefly, a sciatic nerve of a rat was homogenized in a cooled isosmotic solution (250 mM sucrose, 10 mM HEPES-Tris buffer (pH 7.6), 2 mM EDTA, 1 mM PMSF). The homogenate was centrifuged at 4° C. and 3,000 rpm for 10 minutes, and the supernatant was collected and further centrifuged for 45 minutes at 45,000 rpm. The supernatant was removed, and the precipitates were suspended in 100 μl of a 250 mM sucrose solution (dissolved in 10 mM HEPES-Tris buffer (pH 7.6)).

Measurement of Na,K-ATPase Activity

Na,K-ATPase activity was measured by a previously reported method (see Non-Patent Document 6). Briefly, in order to measure a sodium/potassium-dependent activity, there was used a solution for measurement of the Na,K-ATPase activity (0.2 ml) with a composition of 10 mM MgCl₂, 20 mM HEPES-Tris (pH 7.0), 120 mM NaCl, 30 mM KCl, 0.5 mg/ml crude membrane protein, and 25 mM [γ-³²P]ATP (10,000 cpm). A measurement solution with another composition was prepared by adding 1 mM ouabain to the above-mentioned solution. The ouabain-sensitive Na,K-ATPase activity was determined by calculating a difference between a value of the sodium/potassium-dependent activity and a value of the ouabain-sensitive activity. Both the mixed solutions for measurement were incubated at 37° C. for 15 minutes, and then 0.1 mg/ml activated carbon was added, followed by centrifugation at 15,000 rpm for 15 minutes. The supernatants were collected, and radioactivity of inorganic ³²P was measured using a scintillation counter.

Statistical Analysis

Multiple comparisons among the feed groups of the diabetic rats arid the feed groups of the normal rats were carried out by one-way analysis of variance (ANOVA), and statistical differences were evaluated by the Tukey test for the parametric case or by the Kruskal-Wallis test for the nonparametric case. A p-value equal to or less than 0.05 was considered to be statistically significant. Moreover, two-group comparison analyses were carried out between group DC and the other feed groups in the diabetic rat group (or between group C and the other feed groups in the normal rat group) (by the Dunnett's test for the parametric case or by the Dunn's test for the nonparametric case)). Data are expressed as means±standard errors of the means (SEM).

(Results)

The NCVs and Na,K-ATPase activities in the diabetic rats and normal rats were shown in Table 1 as means±standard errors of the means. The NCV in the diabetic rats fed with the control feed (group DC) was found to be significantly lower than that in the normal rats fed with the control feed (group C). The values of the NCVs, in all the experimental rats including both diabetic rats and normal rats, were found to significantly correlate with ouabain-sensitive Na,K-ATPase activities in sciatic nerve membrane fractions obtained from the individuals (correlation coefficient r=0.835, n=24, Table 1, the results were not shown). The results revealed that the ouabain-sensitive Na,K-ATPase activities, instead of NCVs, could be used for evaluation of the degree of a peripheral neuropathy. The NCV value of group DPR was higher than those of groups DWR and DBR (p<0.05), so that a peripheral neuropathy was considered to be improved. The Dunnett's test showed that the Na,K-ATPase activity of group DPR was significantly different (p<0.01) from that of the group of diabetic rats fed with the control feed (group DC).

(Discussion)

The experiments by the inventors of the present invention clarified that diabetic neuropathy was induced five weeks after the STZ treatment, which was confirmed by decreases in NCV values, decreases in ouabain-sensitive Na,K-ATPase activities, etc. It has been reported that a value of Na,K-ATPase activity statistically correlates with a value of NCV and can be used as an alternative method for NCV measurement to estimate clinical severities of peripheral motor nerves (see Non-Patent Document 7), and the inventors of the present invention have achieved the same results. The results revealed that, in the experimental system of the present invention, a value of ouabain-sensitive Na,K-ATPase activity can be used for evaluation of the degree of a peripheral motor neuropathy instead of a value of NCV. This is advantageous in that many samples can be treated rapidly.

[Table 1] Body weight, blood glucose level, and measured value in sciatic nerve or serum of normal or diabetic rats fed with different rice feeds (the values were measured after three weeks)

TABLE 1

| | Treatment | | | |
|---|---|---|---|---|
| | Non-diabetic | | | |
| | Diet | | | |
| | C | WR | BR | PR |
| | Group | | | |
| | C group | WR group | BR group | PR group |
| N | 4 | 6 | 6 | 6 |
| (number of animals) | | | | |
| Weight (g) | 373.8 ± 7.8^{a} | 374.7 ± 3.8^{a} | 370.3 ± 9.1^{a} | 384.8 ± 13.5^{a} |
| Glucose (mg/dl) | 127.0 ± 1.8^{a} | 135.5 ± 2.7^{a} | 129.0 ± 3.3^{a} | 126.7 ± 4.0^{a} |
| NCV (m/s) | 50.4 ± 0.8 | — | — | — |
| ATPase (umol/g/h) | 6102.5 ± 218.9^{ab} | 4855 ± 463.4^{bc} | 6998 ± 515.6^{a} | 6302 ± 371.5^{a} |
| HTase/HDL | 9.8 ± 0.4^{a} | 10.0 ± 0.2^{a} | 10.2 ± 0.3^{a} | 10.3 ± 0.3^{a} |
| (nmol/mg/min) | | | | |

| | Treatment | | | |
|---|---|---|---|---|
| | Diabetic | | | |
| | Diet | | | |
| | C | WR | BR | PR |
| | Group | | | |
| | DC group | DWR group | DBR group | DPR group |
| N | 4 | 9 | 9 | 9 |
| (number of animals) | | | | |
| Weight (g) | 174.0 ± 8.6^{b} | 174.2 ± 4.8^{b} | 183.9 ± 2.7^{ab} | 209.0 ± 3.3^{a*} |
| Glucose (mg/dl) | 438.0 ± 11.0^{a} | 431.0 ± 9.3^{a} | 405.2 ± 9.1^{a} | 351.9 ± 6.3^{b*} |
| NCV (m/s) | 34.0 ± 1.4^{b} | 34.8 ± 0.6^{b} | 37.1 ± 0.5^{b} | 44.4 ± 1.0^{a*} |
| ATPase (umol/g/h) | 2238 ± 102^{b} | 2229.3 ± 153.0^{b} | 2437.2 ± 189.9^{b} | 3867.8 ± 177.6^{a*} |
| HTase/HDL | 2.0 ± 0.3^{c} | 1.8 ± 0.2^{c} | 4.3 ± 0.2^{b**} | 8.9 ± 0.2^{a**} |
| (nmol/mg/min) | | | | |

The abbreviations C, WR, BR, PR, DC, DWR, DBR, and DPR represent groups of normal rats fed with a control feed, normal rats fed with a white rice feed, normal rats fed with a brown rice feed, normal rats fed with a pre-germinated brown rice feed, diabetic rats fed with a control feed, diabetic rats fed with a white rice feed, diabetic rats fed with a brown rice feed, diabetic rats fed with a pre-germinated brown rice feed, respectively,
n: number of samples;
Weight: body weight:
Glucose: glucose concentration;
NCV: nerve conduction velocity;
ATPase: ATPase activity value;
HTase/HDL: HTase activity value per HDL unit quantity (1 mg of a protein).
The values were expressed as means ± standard errors of the means.
Means of the values indicated by different characters (a, b) in the same line were found to have significant differences from each other (P < 0.05).
The values indicated by the symbol “*” were P < 0.01 compared to group DC.
The values indicated by the symbol “**” were P < 0.001 compared to group DC.

### REFERENTIAL EXAMPLE 3

Measurement and Analysis for Comparing Peripheral Nerve Shapes of Rats Fed with Pre-Germinated Brown Rice, Brown Rice, or White Rice

(Experimental Materials and Methods)

Morphometric Analysis

After completion of the animal experiment, a rat was killed and dissected, and the right sciatic nerve was collected from the rat and immersed in a fixative overnight. The nerve was washed with cacodylate buffer (pH 7.2) three times and cut into two pieces, and they were embedded in an epoxy resin (Poly/Bed812, Polysciences Inc., Warrington, Pa.). Slices cross-sectional to the nerve axon were prepared and stained with 1% toluidine blue, followed by observation under an Axiphot light microscope equipped with Axicam (Carl Zeiss, Jena, Germany). The preserved images were analyzed by Axio Vision. The total number of myelinated fibers in each nerve fascicle was visually identified and counted. For myelinated fibers, both the diameter of the axon and the diameters of the entire fiber were measured. Each diameter was calculated as a mean of the major axis and the minor axis. The myelinated fiber diameter, axon diameter, and G ratio (axon diameter calculated by axon diameter/myelinated fiber diameter) were determined and variations in size frequencies of myelinated fibers and axons and G ratios were shown as histograms showing each distribution.

(Results)

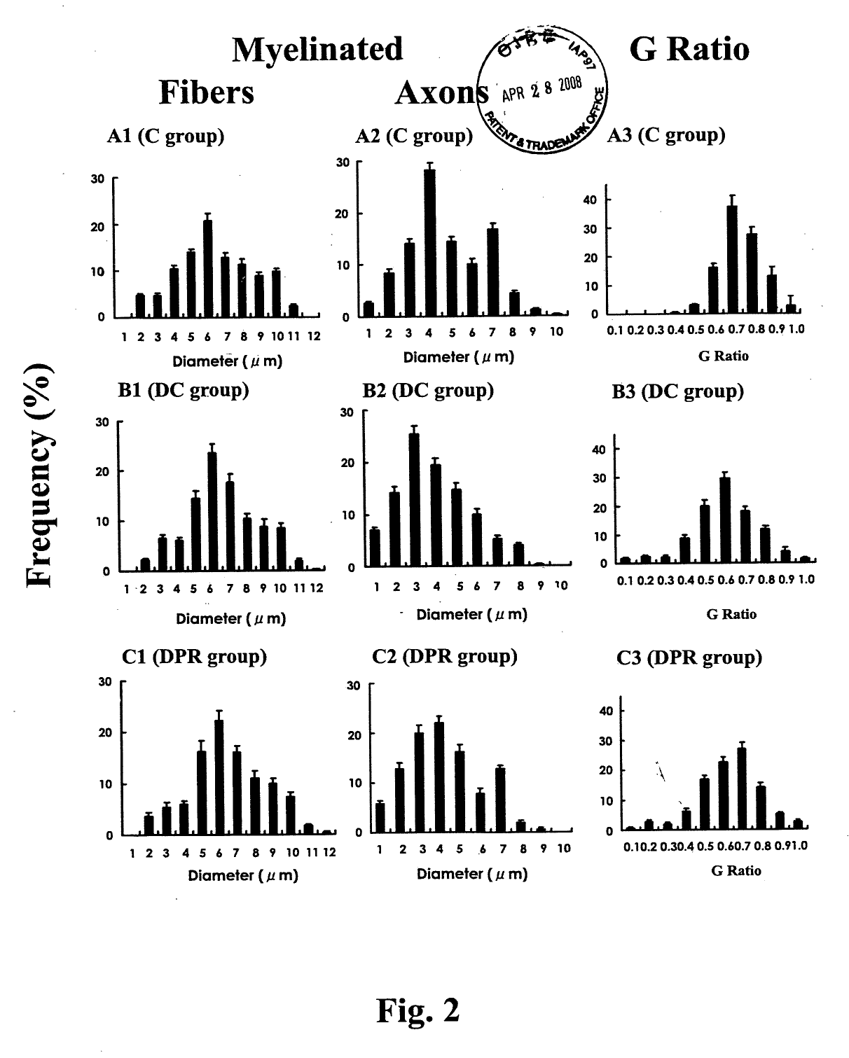

Histograms showing size frequency distributions of X diameters of myelinated fibers in rat sciatic nerves, diameters of myelinated axons in rat sciatic nerves, and G ratios (indexes of degrees of myelination) were compared between group DPR and group C or DC (FIG. 2). There were no differences in the G ratio and diameters of myelinated fibers and axons among individuals and groups due to shifting or sectioning in comparisons of size distributions.

The size distribution of the myelinated fibers was unimodal (had one peak) with a peak at 6.0 μm, and there were no statistically significant differences among three groups.

The diameter distribution of the myelinated axons of the group of diabetic rats fed with pre-germinated brown rice (group DPR) was bimodal (had two peaks) with peaks at 4.0 and 7.0 μm. The mode of distribution was similar to that of the group of normal rats fed with a control feed (group C) and was significantly different from that of the group of diabetic rats fed with a control feed (group DC), which was clearly deformed to the left side. The mode of distribution was similar to that of group C.

The size distributions of the G ratios (which represent degrees of myelination) were unimodal in all the three groups, but shifting of the peaks of groups C, DC, and DPR were observed between 0.7 and 0.6. In the case of group DPR, the distribution of the G ratio was unimodal with a peak at 0.7. The mode of distribution was similar to that of group C.

(Discussion)

It has been reported that abnormalities of peripheral nerve fibers involving axonal degeneration and demyelination (myelin damage) appear in sciatic nerves (see Non-Patent Document 8) and diaphragmatic nerves (see Non-Patent Document 9) of STZ-diabetic rats with chronic hyperglycemia. However, the morphometric analysis in this test clarified that STZ-diabetic rats have damages mainly in the axons and have few damages in the myelins. That is, there are no significant differences in myelin distributions between group C and group DC, while there are differences in axon distributions. Therefore, the fact revealed that the STZ-diabetic rat models were affected mainly in the axons.

The distribution of the diameters of the axons and the G ratio (which represents a degree of myelination) in the diabetic rats fed with a pre-germinated brown rice feed were found to be similar to those of normal rats. These findings show that ingestion of pre-germinated brown rice may prevent or repair damages in myelinated axons of STZ rat models with diabetic neuropathy.

### REFERENTIAL EXAMPLE 4

Experiment for Comparison of HTase Activities of Rats Fed with Pre-Germinated Brown Rice, Brown Rice, or White Rice

(Experimental Materials and Methods)

HTase Measurement

The activity of HTase in rat serum HDL was measured using a commercially available measurement kit (Alfresa Auto HTLase; Alfresa Pharma Corp., Osaka, Japan). This kit uses γ-thiobutyrolactone as a substrate. HTase hydrolyzes a lactone ring of the substrate to generate a free thiol group. The thiol group reacts with 5,5′-dithiobis(2-nitrobenzoic acid) to generate 5-thio-2-nitrobenzoic acid, which was measured by the absorbance at 450 nm. The absorbance at 450 nm was measured to calculate the enzymatic activity.

Statistical Analysis

Multiple comparisons among the feed groups of the diabetic rats and the feed groups of the normal rats were carried out by one-way analysis of variance (ANOVA), and statistical differences were evaluated by the Tukey test for the parametric case or by the Kruskal-Wallis test for the nonparametric case. A p-value equal to or less tan 0.05 was considered to be statistically significant. Moreover, comparative analyses of two groups were carried out between group DC and the other feed groups in the diabetic rat group (or between group C and the other feed groups in the normal rat group) (by the Dunnett's test for the parametric case or by the Dunnett's test for the nonparametric case)). Data were expressed as means±standard errors of the means (SEM).

(Results)

The concentrations or activity values of HTase in serum of diabetic rats and normal rats were shown in Table 1 as means±standard errors (SEM).

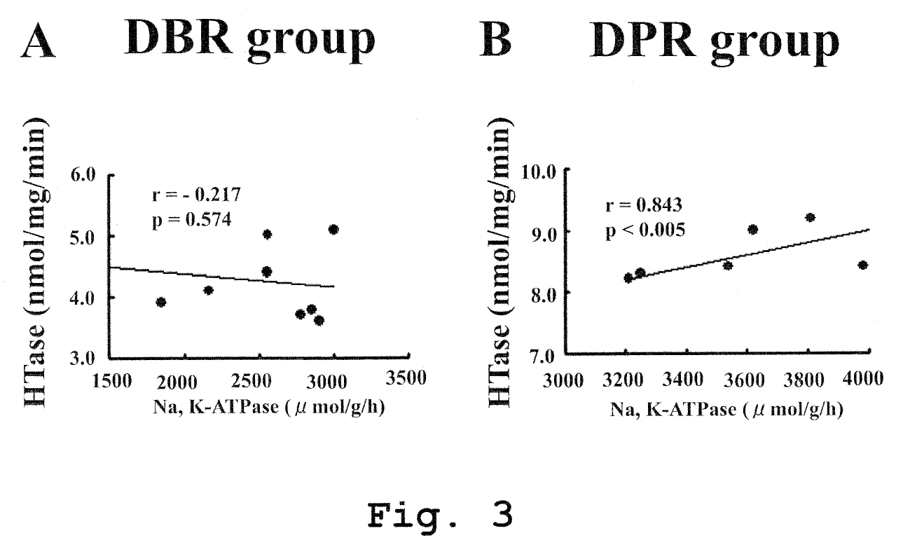

As previously reported (see Non-Patent Document 10), the activity of HTase in serum HDL of the diabetic rats was found to decrease. The decrease in HTase activity of the diabetic rats fed with a pre-germinated brown rice feed (group DPR) was found to be suppressed compared to the rats fed with a control feed (AIN93G) (group DC), as shown by statistically significant differences (Table 1). A correlation analysis of the diabetic rat group fed with a brown rice feed (group DBR) and group DPR revealed that the HTase activity of group DPR correlates with the Na,K-ATPase activity, while the HTase activity of group DBR does not correlate with the Na,K-ATPase activity (FIG. 3).

(Discussion)

### EXAMPLE 1

Experiment for Comparison of Effects By Addition of Total Lipid Fraction on Activity of Na,K-ATPase Derived from Rat Sciatic Nerve Membrane

As described in “Description of the Related Art”, pre-germinated brown rice produces γ-aminobutyric acid (GABA), which is known to have an antihypertensive action and an antistress action, in germination process. A lipid fraction of pre-germinated brown rice is known to contain the GABA and various effective ingredients that are not present in brown rice. Note that the ingredient having the effect of improving a neuropathy of the present invention is not the GABA at least as a single molecular species (data were not shown). Therefore, the inventors of the present invention focused attention on a total lipid fraction derived from pre-germinated brown rice and brown rice (hereinafter, a total lipid fraction derived from pre-germinated brown rice and brown rice are referred to as TLp and TLb, respectively), and examined whether or not each fraction has an effect of improving a peripheral neuropathy. As shown in Referential Example 2, in order to evaluate the degree of a peripheral neuropathy, the ouabain-sensitive Na,K-ATPase activity can be used instead of NCV Therefore, an effect of total lipid fractions on the ouabain-sensitive Na,K-ATPase activity was examined.

(Experimental Materials and Methods)

Separation of Lipoproteins

Lipoproteins were obtained in accordance with a method based on a previously reported procedure (see Non-Patent Document 11). Briefly, fresh serums obtained from normal rats were collected, and the density was adjusted with solid KBr to 1.3 g/ml. Normal physiological saline (3.5 ml, 1.006 g/ml) was layered on the thus-prepared serum (1.5 ml, 1.3 g/ml), followed by discontinuous density gradient ultracentrifugation in a centrifuge tube. Lipoproteins were separated by ultracentrifugation at 369,548 g and 4° C. for 45 minutes in a TV865 rotor. Three kinds of main lipoprotein fractions (VLDL, LDL, and HDL) were collected and dialyzed against PBS at 4° C. overnight. In the present specification, the terms “LDL” and “HDL” refer to the respective fractions obtained by this method.

Preparation of Total Lipid Fraction

A total lipid fraction (TLp or TLb) was obtained by double extraction of 5 g of pre-germinated brown rice or brown rice with 30 ml and 20 ml of chloroform/methanol (1:1 and 2:1, volume ratios).

Reaction of Hcy-Thiolactone and Low-Density Lipoproteins (LDL)

In vitro Hcy-thiolactonization of LDL was carried out under previously reported experimental conditions (see Non-Patent Document 12). Briefly, an appropriate amount of LDL solution (containing 100 μg of LDL proteins) was suspended in 10 mM PBS (pH 8.2), and the suspension was incubated with Hcy-thiolactone (100 μmol/L) and indicated amounts (from 0.1 to 1.0 μg) of a total lipid fraction (TLp or TLb) at 37° C. for two hours while stirring gently. After incubation, the mixed solution was passed through Bio-gel P-2 column equilibrated with 10 mM PBS (pH 8.2) to remove unreacted Hcy-thiolactone.

Preparation of Crude Sciatic Nerve Membrane for Measurement of Na,K-ATPase Activity

The crude membrane was prepared by a previously reported procedure (see Non-Patent Document 6). Briefly, a sciatic nerve of a rat was homogenized in a cooled isosmotic solution (250 mM sucrose, 10 mM HEPES-Tris buffer (pH 7.6), 2 mM EDTA, 1 mM PMSF). The homogenate was centrifuged at 4° C. and 3,000 rpm for 10 minutes, and the supernatant was collected and further centrifuged for 45 minutes at 45,000 rpm. The supernatant was removed, and the precipitates were suspended in 100 μl of a 250 mM sucrose solution (dissolved in 10 mM HEPES-Tris buffer (pH 7.6)).

Measurement of Na,K-ATPase Activity

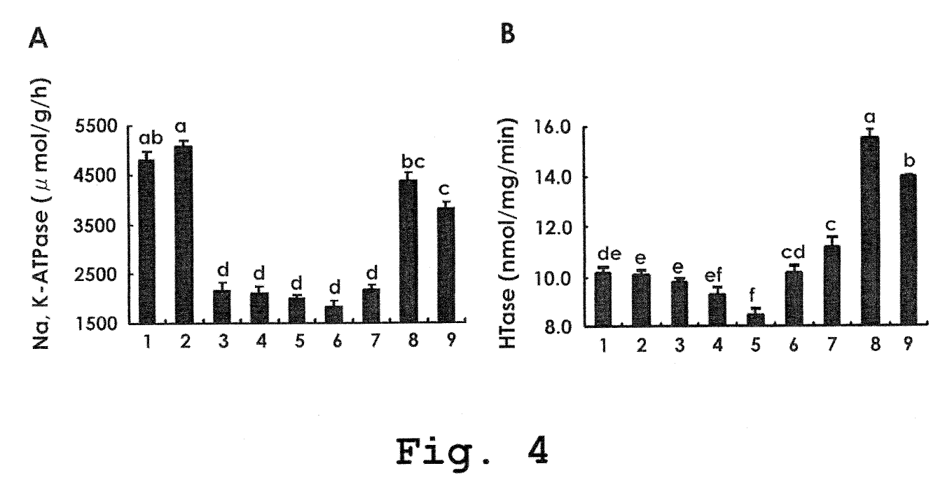

Na,K-ATPase activity was measured by a previously reported method (see Non-Patent Document 6). Briefly, in order to measure a sodium/potassium-dependent activity, there was used a solution for measurement of the Na,K-ATPase activity (0.2 ml) with a composition of 10 mM MgCl₂, 20 mM HEPES-Tris (pH 7.0), 120 mM NaCl, 30 mM KCl, 0.5 mg/ml crude membrane protein, and 25 mM [γ-³²P]ATP (10,000 cpm). A measurement solution with another composition was prepared by adding 1 mM ouabain to the above-mentioned solution. The ouabain-sensitive Na,K-ATPase activity was determined by calculating a difference between a value of the sodium/potassium-dependent activity and a value of the ouabain-sensitive activity. Both the mixed solutions for measurement were incubated at 37° C. for 15 minutes, and then 0.1 mg/ml activated carbon was added, followed by centrifugation at 15,000 rpm for 15 minutes. The supernatants were collected, and radioactivity of inorganic ³²P was measured using a scintillation counter. LDL, Hcy-thiolactone, and TLb or TLp were added to the reaction solutions in this order in amounts described in the brief description of FIG. 4.

Statistical Analysis

Multiple comparisons among the respective groups were carried out by one-way analysis of variance (ANOVA), and then statistical differences were evaluated by the Tukey test. A p-value equal to or less than 0.05 was considered to be statistically significant.

(Results)

A change in the Na,K-ATPase activity in normal rat tissues was examined by incubating LDL modified with Hcy-thiolactone and Hcy-thiolactone together with TLp or TLb (FIG. 4). In a crude sciatic nerve membrane protein sample incubated with Hcy-thiolactone-modified LDL, the Na,K-ATPase activity decreased significantly compared to unmodified LDL (P<0.05) (FIG. 4A bar graph: 2 and 3). When the sample was incubated with TLp, the Na,K-ATPase activity that had been inhibited by Hcy-thiolactone-modified LDL was recovered to some extent (FIG. 4A bar graph: 7, 8, and 9). On the other hand, in the case of TLb, such effects were not observed (FIG. 4A bar graph: 4, 5, and 6). None of TLp or TLb alone did not affect on the Na,K-ATPase activity (data were not shown).

(Discussion)

The inventors of the present invention examined enzymatic activities in total lipid fractions extracted from bran of pre-germinated brown rice and brown rice (TLp and TLb) to investigate whether or not pre-germinated brown rice contains any factors having an excellent effect of improving a peripheral neuropathy compared to brown rice. It has been reported that Hcy-thiolactone-modified LDL decreases the Na,K-ATPase activity in cultured human aortic endothelial cells (see Non-Patent Document 12). Therefore, the inventors of the present invention presumed that a decrease in the Na,K-ATPase activity in diabetic neuropathy occurs via modification of LDL with Hcy-thiolactone. The inventors of the present invention have discovered that Hcy-thiolactone-modified LDL decreases the Na,K-ATPase activity (FIG. 4A bar graph: 2 and 3) even in biomaterials derived from a sciatic nerve of a normal rat. In the experiment by the inventors of the present invention, in the case of adding TLp (from 0.1 to 10 μg) to Hcy-thiolactone-modified LDL, the Na,K-ATPase activity decreased slightly, while in the case of adding TLb to Hcy-thiolactone-modified LDL, the Na,K-ATPase activity decreased in the same way as in the case of Hcy-thiolactone-modified LDL. Single use of TLp or TLb did not affect on the Na,K-ATPase activity (data were not shown). The results indicate that TLp contains some inhibitor(s) that eliminates or decreases an effect on the Na,K-ATPase activity in Hcy-thiolactone-modified LDL.

The inventors of the present invention continue to study to identify a specific ingredient that is contained in pre-germinated brown rice and provides an effect of improving a neuropathy. In the future, they plan to further fractionate a total lipid fraction to identify a single substance or a relatively small number of substances that acts cooperatively with each other. In addition, they plan to make he similar study using biomaterials derived from a peripheral nerve system of a diabetic rats, and ultimately to make a study using the identified single substance or relatively small number of substances that acts cooperatively with each other in diabetic human or animal individuals. Moreover, in the future, they plan to identify a biological molecule targeted by the identified single substance or relatively small number of substances that acts cooperatively with each other.

### EXAMPLE 2

Experiment for Comparison of Effects By Addition of Total Lipid Ingredient on HTase Activity in HDL Derived from Rat Serum

As shown in Referential Example 4, in tie case of diabetic rats, the activity of HTase derived from serum decreased, and only in the case of group DPR, a decrease in the activity was suppressed compared to groups DWR DBR, and DC (Table 1). Meanwhile, in the case of group DPR, the activity of Na,K-ATPase derived from a sciatic nerve membrane was found to correlate with the activity of HTase derived from rat serum (FIG. 3). Therefore, the inventors of the present invention focused attention on the HTase activity in HDL to clarify action mechanisms of effects of improving a neuropathy observed in Referential Examples 1 to 4 and an effect of suppressing a decrease in the Na,K-ATPase activity observed in Example 1. The inventors of the present invention examined whether or not TLp has an ability to suppress a decrease in the HTase activity or to increase the activity compared to TLb. HTase is present in serum HDL and plays an important role in antioxidation of LDL.

(Experimental Materials and Methods)

Separation of Lipoproteins

Lipoproteins were obtained in accordance with a method based on a previously reported procedure (see Non-Patent Document 11). Briefly, fresh sera obtained from normal rats were collected, and the density was adjusted with solid KBr to 1.3 g/ml. Normal physiological saline (3.5 ml, 1.006 g/ml) was layered on the thus-prepared serum (1.5 ml, 1.3 g/ml), followed by discontinuous density gradient ultracentrifugation in a centrifuge tube. Lipoproteins were separated by ultracentrifugation at 369,548 g and 4° C. for 45 minutes in a TV865 rotor. Three kinds of main lipoprotein fractions (VLDL, LDL, and HDL) were collected and dialyzed against PBS at 4° C. overnight. In the present specification, the terms “LDL” and “HDL” refer to the respective fractions obtained by this method.

Preparation of Total Lipid Fraction

A total lipid fraction (TLp or TLb) was obtained by double extraction of 5 g of pre-germinated brown rice or brown rice with 30 ml and 20 ml of chloroform/methanol (volume ratio, 1:1 and 2:1).

HTase Activity Measurement

The activity of HTase in rat serum HDL was measured using a commercially available measurement kit (Alfresa Auto HTLase; Alfresa Pharma Corp., Osaka, Japan). This kit uses γ-thiobutyrolactone as a substrate. HTase hydrolyzes a lactone ring to generate a free thiol group. The thiol group reacted with 5,5′-dithiobis(2-nitrobenzoic acid) to generate 5-thio-2-nitrobenzoic acid, which was measured by the absorbance at 450 nm. The absorbance at 450 nm was measured to calculate the enzymatic activity. TLp and TLb were added to the reaction solution in amounts described in the brief description of FIG. 5.

Statistical Analysis

Multiple comparisons among the respective groups were carried out by one-way analysis of variance (ANOVA), and then evaluated by the Tukey test. A p-value equal to or less than 0.05 was considered to be statistically significant.

(Results)

HDL prepared from serum of normal rats was used as an HTase source to examine whether or not TLp or TLb affects on the HTase activity. In the case of incubation together with TLp, the HTase activity was found to be significantly different (P<0.05) from that in the case of incubation together with TLb or in the case of adding neither TLp nor TLb (FIG. 4B bar graph: 6, 7, 8, and 9). TLp exhibited a dosage-dependent (from 0.1 to 1.0 μg) HTase activity-promoting effect, and the effect reached plateau at 5 μg. On the other hand, TLb did not exhibit the effect on the HTase activity in the same range (FIG. 4B bar graph: 2, 3, 4, and 5).

(Discussion)

The inventors of the present invention showed that TLp directly enhances the HTase activity in HDL. This suggested that an effect of suppressing a decrease in the Na,K-ATPase activity by TLp could be provided through an action on an molecule with HTase activity in HDL. However, an effective ingredient in TLp is not necessarily a single ingredient, and a target molecule of the effective ingredient is not necessarily a single molecule.

Meanwhile, this example revealed that ingestion of TLp could improve the decrease in HTase activity in HDL, which is one of symptoms of diabetic neuropathy.

### REFERENCES

[Non-Patent Document 6] Silva I V, Caruso-Neves C, Azeredo I M, Carvalho T L, Lara L S, de Mello M C, Lopes A G. Urea inhibition of renal (Na⁺+K⁺)ATPase activity is reversed by cAMP. Arch Biochem Biophys. Oct. 15, 2002;406(2):183-9.
[Non-Patent Document 7] Andersen H, Nielsen J F, Nielsen V K. Inability of insulin to maintain normal nerve function during high-frequency stimulation in diabetic rat tail nerves. Muscle Nerve. January 1994;17(1):80-4.
[Non-Patent Document 8] Sugimoto K, Yagihashi S. Peripheral nerve pathology in rats with streptozotocin-induced insulinoma. Acta Neuropathol (Berl). 1996;91(6):616-23.
[Non-Patent Document 9] Rodrigues Filho O A, Fazan V P. Steptozotocin induced diabetes as a model of phrenic nerve neuropathy in rats. J Neurosci Methods. Mar. 15, 2006;151(2):131-8. Epub Aug. 25, 2005.
[Non-Patent Document 10] Kosaka T. Yamaguchi M, Motomura T, Mizuno K. Investigation of the relationship between atherosclerosis and paraoxonase or homocysteine thiolactonase activity in patients with type 2 diabetes mellitus using a commercially available assay. Clin Chim Acta. September 2005;359(1-2):156-62.
[Non-Patent Document 11] Chung B H, Wilkinson T, Geer J C, Segrest J P. Preparative and quantitative isolation of plasma lipoproteins: rapid, single discontinuous density gradient ultracentrifugation in a vertical rotor. J Lipid Res. March 1980;21(3):284-91.
[Non-Patent Document 12] Vignini A, Nanetti L, Bacchetti T, Ferretti G, Curatola G, Mazzanti L. Modification induced by homocysteine and low-density lipoprotein on human aortic endothelial cells: an in vitro study. J Clin Endocrinol Metab. September 2004;89(9):4558-61.

### INDUSTRIAL APPLICABILITY

A total lipid fraction in pre-germinated brown rice of the present invention has an effect of preventing or improving diabetic neuropathy.

Therefore, according to the present invention, the fraction can be used for an agent for prevention or improvement of diabetic neuropathy.

Pre-germinated brown rice has been conventionally used as a health food, and therefore it is very safe, cm be ingested continuously, produced in a large quantity, and easily added to foods or the like. Accordingly, pre-germinated brown rice is likely to contribute to health promotion and disease prevention for human or animals.

What is claimed is:

1. An agent for prevention or improvement of a neuropathy, comprising a pre-germinated brown rice lipid fraction as an effective ingredient.

2. An agent according to claim 1, wherein the pre-germinated brown rice lipid fraction comprises a chloroform-methanol soluble component of pre-germinated browm rice.

3. An agent according to claim 1, wherein the neuropathy comprises diabetic neuropathy.

4. An agent according to claim 1, wherein the neuropathy comprises damage of myelinated nerves.

5. An agent according to claim 1, wherein the neuropathy comprises a decrease in activity of Na,K-ATPase derived from the nerve membrane.

6. An agent according to claim 1, wherein the neuropathy is accompanied by a decrease in activity of HTase in HDL derived from serum.

7. A functional food for one of prevention and improvement of a neuropathy, comprising a pre-germinated brown rice lipid fraction as an effective ingredient.

8. A functional food according to claim 7, wherein the pre-germinated brown rice lipid fraction comprises a chloroform-methanol soluble component of pre-germinated brown rice.

9. A functional food according to claim 7, wherein the neuropathy comprises diabetic neuropathy.

10. A functional food according to claim 7, wherein the neuropathy comprises damage of myelinated nerves.

11. A functional food according to claim 7, wherein the neuropathy comprises a decrease in activity of Na,K-ATPase derived from the nerve membrane.

12. A functional food according to claim 7, wherein the neuropathy is accompanied by a decrease in activity of HTase in HDL derived from serum.

13. A method for one of prevention and treatment of a neuropathy, comprising administering a pre-germinated brown rice lipid fraction.

14. A method according to claim 13, wherein the pre-germinated brown rice lipid fraction comprises a chloroform-methanol soluble component of pre-germinated brown rice.

15. A method according to claim 13, wherein the neuropathy comprises diabetic neuropathy.

16. A method according to claim 13, wherein the neuropathy comprises damage of myelinated nerves.

17. A method according to claim 13, wherein the neuropathy comprises a decrease in activity of Na,K-ATPase derived from the nerve membrane.

18. A method according to claim 13, wherein the neuropathy is accompanied by a decrease in activity of HTase in HDL derived from serum.
